(19) United States
(12) Patent Application Publication
(10) Pub. No.: US 2006/0085062 A1
(43) Pub. Date: Apr. 20, 2006

(54) Implantable stent with endothelialization factor

(75) Inventors: Randall J. Lee, Hillsborough, CA (US); Kenneth J. Colley, San Francisco, CA (US); James C. Peacock III, San Carlos, CA (US)

Correspondence Address: PRESTON GATES & ELLIS LLP;ATTN: C. RACHAL WINGER, 925 FOURTH AVE, SUITE 9200, SEATTLE, WA, 98104-1158, US

(73) Assignee: Medlogics Device Corporation, Santa Rosa, CA (US)

(21) Appl. No.: 11/223,741

(22) Filed: Sep. 8, 2005

Related U.S. Application Data
(63) Continuation-in-part of application No. 10/724,453, filed on Nov. 28, 2003.
(60) Provisional application No. 60/607,832, filed on Sep. 8, 2004.

(51) Int. Cl. A61F 2/06

(52) U.S. Cl. 623/1.39

(57) ABSTRACT

A stent is provided in combination with a growth factor, specifically pleiotrophin or an analog or derivative thereof, which promotes endothelialization of the stent and re-endothelialization of the stented region of an injured site in a body lumen. In particular applications, the stent is an endolumenal stent and the growth factor promotes healing via endothelialization and substantially prevents restenosis. The growth factor is delivered from the stent formulated as a protein or peptide, or as a gene transfer vector. Methods for the treatment of vascular injury using pleiotrophin are also disclosed.

### RELATED APPLICATION

This application claims priority to U.S. Provisional Patent Application No. 60/607,832 filed Sep. 8, 2004 and is a continuation-in-part of U.S. Patent Application No. 10/724,453 filed Nov. 28, 2003 which is incorporated by reference herein in its entirety.

### FIELD OF THE INVENTION

This invention relates to implantable medical devices and related methods of manufacture and use. More specifically, it relates to implantable stents. Still more specifically, it relates to an implantable stent coated with a compound that promotes endothelialization of the stented region of the body.

### BACKGROUND OF THE INVENTION

Implantable stents have been under significant development for more than a decade, and many different designs have been investigated and made commercially available for use in providing mechanical scaffolding to hold body lumens open or patent. Stents are generally used in many different body lumens, including, without limitation, blood vessels, the gastrointestinal tract, biliary ducts, fallopian tubes, and the vas deferens. Vascular stents are generally tubular members formed from a lattice of structural struts that are interconnected to form an integrated strut network that forms a wall surrounding an axis. The integrated strut lattice typically includes inter-strut gaps through which the inner luminal axis within the stent wall and the outer region surrounding the stent wall are able to “communicate.” The ability of areas within and outside a stent to “communicate” is beneficial for example when a stent is implanted in an area of a vessel with side branches. The “communication” provided by the inter-strut gaps allows side branches to beneficially receive flow from the main lumen through the inter-strut gaps in the stent wall.

Stents are most frequently used in an interventional recanalization procedure, adjunctive to methods such as balloon angioplasty or atherectomy. Balloon-expandable stents are generally constructed from a material, such as stainless steel or cobalt-chromium alloy for example, that is sufficiently ductile to be delivered in a collapsed condition on the surface of a deflated balloon. The stents are then expanded against the subject lumenal wall by inflation of the balloon and are substantially retained in the expanded condition as an implant upon subsequent balloon deflation. Self-expanding stents are generally constructed of an elastic, super-elastic or shape-memory material, such as nickel-titanium alloys. These materials typically have a memory state that is expanded, but are delivered to the implantation site in a collapsed condition for appropriate delivery profiles. Once in place, the stent is released to recover or self-expand against the lumenal wall where it is then left as the implant.

The majority of commercially available stents form completely integrated tubular structures, with continuity found along the integrated strut lattice both circumferentially as well as longitudinally. In order to provide for the adjustability between the collapsed and expanded conditions, such stents generally incorporate undulating shapes for the struts, which shapes are intended to reconfigure to allow for maximized radial expansion with minimized longitudinal change along the stent length. This is generally desirable for example in order to achieve repeatable, predictable placement of the stent along a desired length of a localized, diseased region to be re-opened, as well as to maintain stent coverage over the expanding balloon at the balloon ends. A stent that substantially shortens during balloon expansion exposes the balloon ends to localized vessel wall trauma at those ends without the benefit of the stent scaffolding to hold those regions open long-term after the intervention is completed.

Notwithstanding the prevalence of the foregoing type of stent, other designs have also been disclosed that either further modify such general structures, or further depart from the basic design. For example, one additional type of stent forms a wall that is not circumferentially continuous, but has opposite ends along a sheet formed from the strut lattice. This sheet is adjusted to the collapsed condition by rolling the stent from one end to the other. At the site of implantation, the stent is unrolled to form the structural wall that radially engages the lumenal wall and substantially around an inner lumen. In the event the stent is undersized to the lumen, the opposite ends overlap and thus double the thickness of implant material that protrudes from the lumen wall and into the lumen.

While stents are typically intended to maintain vessel patency, other uses have been disclosed. For example, some stents have been disclosed for the purpose of occluding the subject lumen where the stent is implanted. Examples of such stents include fibrin coated stents, and examples of such occlusive uses for stents include fallopian tubal ligation and aneurysm closure.

Stents have been further included in assemblies with other structures, such as grafts to form stent-grafts. These assemblies generally incorporate a stent structure that is secured to a graft material, such as a textile or other sheet material. Examples of uses that have been disclosed for stent-grafts include aneurysm isolation, such as along the abdominal aorta wall.

Vascular stents have had an enormous impact upon the occurrence of restenosis following recanalization procedures. Restenosis is a re-occlusion of the acutely recanalized blockage that typically takes place within 3-6 months after intervention, and is generally a combination of mechanical and physiological responses to the vessel wall injury caused by the recanalization procedure itself. In one regard, restenosis can occur at least in part from an elastic recoil of the expanded vessel wall, such as following expansion of the wall during balloon angioplasty. With respect to the physiological response to injury, it has generally been observed that injury from the recanalization to the intimal, medial, and sometimes adventitial layers of a vessel wall causes smooth muscle cells within the wall to undergo aggressive mitosis and hyperproliferation, dividing and migrating into the vessel lumen to form a scar that occludes the vessel lumen. Whereas angioplasty and other recanalization interventions prior to the advent of stenting result in an approximately 30%-50% restenosis rate, stenting has generally reduced this rate to about 20%-30%, which reduction is considered a result of the mechanical prevention of vascular recoil.

Recent efforts in vascular stenting have attempted to incorporate an additional therapy adjunctive to stenting to further reduce the incidence of restenosis. One effort has been to locally deliver therapeutic doses of radiation to the vessel wall concomitant to stenting by incorporating radioactive materials into or on the stent scaffolding itself. However, the use of radioactive materials carries a significant burden peri-operatively in handling and disposal and results have not yet been compelling. Moreover, local energy delivery such as via radioactive stents is substantially different than local elution delivery of materials and compounds from stents which are thereafter subject to diffusion, flow, and other active transport mechanisms.

More recently, a substantial effort has been underway to incorporate local drug delivery to stented lesions specifically to retard and prevent restenosis. For example, various local delivery devices have been disclosed to provide highly localized injection of an anti-restenotic material into the injured wall, such as via micro-needles incorporated onto the outer skin of expandable balloons.

A more substantial effort, however, has been to incorporate the anti-restenosis drug on or into the stents themselves in a manner such that the stent elutes the drug into the vessel wall over a prescribed period of time following implantation. These drug eluting stents (DES) provide a stent scaffold having an outer coating that holds and elutes the drug.

The most prevalent form of coating disclosed for use in DES includes polymers, such as, for example, a two-layer polymer coating with one layer holding drug and another layer retarding elution to provide extended elution of the drug, or with one layer providing adhesion to the underlying stent metal and the other layer holding and eluting the drug. Other examples of DES coatings include ceramics, hydrogels, biosynthetic materials, and metal-drug matrix coating.

Examples of drugs that have been investigated for anti-restenosis uses from DES include anti-mitotics, anti-proliferatives, anti-inflammatory, and anti-migratory compounds. Further examples of compounds previously disclosed for use in DES devices and methods include: angiotensin converting enzyme (ACE) inhibitors, angiotensin receptor antagonists, anti-sense materials, anti-thrombotics, platelet aggregation inhibitors, iron chelators (e.g. exochelin), everolimus, tacrolimus, vasodilators, nitric oxide, and nitric oxide pressors or promoters.

Two more specific compounds that have been under substantial clinical investigation on DES include rapamycin (sirolimus) and Taxol® (paclitaxel). Drug eluting stents which incorporate these two compounds have made substantial strides toward reducing restenosis rates in stented lesions from about 20%, to a reduced rate around 10%, and possibly lower with respect to certain patient sub-populations.

Notwithstanding the substantial improvements that appear to be anticipated in view of the recent sirolimus and paclitaxel DES clinical experiences, however, various needs still remain and are believed to be unmet by these and other previously disclosed DES efforts. Therefore, it may be possible to lower the restenosis rate further with drugs having increased potency or other mechanisms of action. However, concerns remain regarding other possible harmful efforts of DES approaches which interfere with the smooth muscle cell cycle such as toxicity, weakening of the vessel lining, late loss, negative remodeling, and possible aneurysm formation.

One approach for preventng restenosis is to promote re-endothelialization of the injured region of lumen where the stent is implanted and thereby to promote vascular wound healing. During stent placement in blood vessels, the vessel injury that typically initiates the cascade of events of the restenosis cycle includes denudation of the endothelium along the vessel lining. Endothelium lines the vessel wall and provides, among other things, a barrier between the smooth muscle cell lining of the vessel wall and various factors within the blood pool of the inner lumen. Once denuded of the endothelium, and frequently also concomitant with breaking of an elastic lamina barrier between the endothelium and media/adventitia, these factors are exposed to the muscle cells and initiate the restenosis cascade as pressors to mitosis, migration, and hyper-proliferation into the vessel. Accordingly, promoting re-endothelialization, and hence re-establishing the barrier against the restenosis pressors from the blood pool, has been promoted as a viable, less traumatic, and highly advantageous approach to preventing restenosis. Moreover, by preventing restenosis by promoting re-endothelialization, many side effects concomitant with various cytotoxic or other anti-proliferative agent approaches are avoided, including for example weakening of the wall, negative remodeling, and possible aneurysm formation.

One example of this re-endothelialization approach intended to treat restenosis includes delivering vascular endothelial growth factor (VEGF) to promote endothelialization of an injured vessel wall. Another example intended to promote re-endothelialization over a stent provides antibodies on a stent surface which are intended to attract adhesion of endothelium. None of these approaches have been shown to provide sufficient safety and efficacy in preventing restenosis to be advanced to widespread commercial use. Therefore successful approaches to. promoting re-endothelialization of stented vessels would provide substantial benefit to patients.

Pleiotrophin (PTN) is a growth factor that has been previously investigated for promoting angiogenesis and has also been observed as a potent agent to promote self-limiting endothelial cell proliferation, and may be useful for wound healing applications. However, incorporation of this growth factor with vascular stents for vascular wound healing, e.g. endothelialization of vascular or other lumen linings to heal wall injury and prevent restenosis, has yet to be disclosed.

Therefore there exists a need for a potent, safe, and efficacious compound, such as pleiotrophin, which can be associated with a stent, to promote endothelialization of the stent, and re-endothelialization of the denuded stented region, in a manner that is safe and substantially prevents restenosis.

### SUMMARY OF THE INVENTION

The present invention provides a medical device having a growth factor releasable disposed on the surface such that the growth factor promotes endothelialization of the device. The medical device of the present invention, such as an endolumenal stent, has a porous outer surface on the substrate that includes a plurality of pores wherein the pores contain a bioerodable or biodegradable material in combination with a bioactive agent, such as a growth factor. The stent is adapted to be positioned at a treatment site such that the pores are exposed to a body of a patient and the growth factor is released from the porous outer surface and promotes endothelialization of the treatment site.

The present invention provides an implantable endolumenal stent system having a bioerodable material in combination with a bioactive agent. The endolumenal stent is adapted to be implanted at a treatment site within a lumen of a body of a patient such that the scaffold engages a luminal wall that defines the lumen and such that the porous outer surface is exposed to at least one biological fluid within the lumen. At the treatment site, the composite material is adapted to bioerode or biodegrade such that the bioactive agent is released from the porous outer surface and the porous outer surface is left remaining on the scaffold.

A relatively passive, or non-reactive, external surface coating on implantable medical devices is provided such that following release of bioactive agents therefrom, there is no reaction to the device-tissue interface in long-term implants.

In yet another embodiment of the present invention, a medical device is provided with an electrolessly electrochemically deposited porous outer surface adapted to exhibit substantially robust adherence to the underlying implantable device substrate thereby providing surface integrity through delivery and in vivo use of the medical device, and is adapted to carry and elute at least one bioactive agent.

In an embodiment of the present invention, a method of coating a stent is provided comprising an electroless electrochemical bath with particles suspended therein, in which the particles comprise a bioactive agent in combination with a bioerodable or biodegradable material. The bath is adapted to electrolessly electrochemically deposit a metal composite matrix onto a activated surface, and also to co-deposit the particles within pores formed within the metal composite matrix.

In one embodiment of the present invention an implantable endolumenal stent system is provided comprising a scaffold with a porous outer surface having a plurality of pores and a composite material located within each of the pores and comprising a bioerodable material in combination with a bioactive agent. In another embodiment of the endolumenal stent system of the present invention, the composite material comprises a plurality of particles wherein the particles comprise an outer diameter that is less than about 5 microns and wherein the particles comprise a bioerodable polymer in combination with the bioactive agent.

In yet another embodiment of the endolumenal stent system of the present invention, the porous outer surface comprises a material that is not inherently porous; and the pores are formed within the material. In various embodiments, the pores are laser cut, photochemically etched or chemically etched into the material. In another embodiment, the porous outer surface comprises a sintered material.

In another embodiment of the endolumenal stent system of the present invention, the endolumenal stent comprises a scaffold constructed from a first material, the porous outer surface comprises a coating material located on the first material, and the pores are located within the coating material. In one embodiment the coating material comprises a non-polymeric material such as an electrochemically deposited material. In another embodiment, the electrochemically deposited material comprises an electrolessly electrochemically deposited material.

In yet another embodiment of the endolumenal stent system of the present invention, the electrolessly electrochemically deposited material comprises a composite material with a metal and a reducing agent of the metal wherein the metal is selected from the group consisting of nickel and cobalt and the reducing agent is phosphorous.

In another embodiment of the endolumenal stent system of the present invention, the first material is selected from the group consisting of stainless steel alloys, nickel-titanium alloys and cobalt-chromium alloys.

In yet another embodiment of the endolumenal stent system of the present invention, the bioactive agent comprises a growth factor wherein the growth factor is pleiotrophin, or an analog or derivative thereof.

In an embodiment of the endolumenal stent system of the present invention, the ratio of bioactive agent to bioerodable material in the composite material is at least about 5:1 and wherein said bioerodable material comprises a bioerodable polymer material.

A relatively passive, or non-reactive, external surface coating on implantable medical devices is provided such that following release of bioactive agents therefrom, there is no reaction to the device device-tissue interface in long-term implants.

In yet another embodiment of the present invention, a medical device is provided with an electrolessly electrochemically deposited porous outer surface adapted to exhibit substantially robust adherence to the underlying implantable device substrate thereby providing surface integrity through delivery and in vivo use of the medical device, and is adapted to carry and elute at least one bioactive agent.

In an embodiment of the present invention, a method of coating a stent is provided comprising an electroless electrochemical bath with particles suspended therein, in which the particles comprise a bioactive agent in combination with a bioerodable or biodegradable material. The bath is adapted to electrolessly electrochemically deposit a metal composite matrix onto a activated surface, and also to co-deposit the particles within pores formed within the metal composite matrix.

In another embodiment of the present invention, a method is provided by which a growth factor, such as pleiotrophin, or an analog or derivative thereof, is delivered to a treatment site, such as an injured region of a luminal wall in order to promote healing or prevent restenosis of the injured region of the luminal wall.

In an embodiment of the present invention, a method is provided for delivering a growth factor to an injured region of a body lumen such that endothelialization of the injured region is promoted and substantial occlusion of the body lumen is prevented.

In one embodiment of the present invention, a method for induction of vascular wound healing by induction of endothelialization of a vascular lesion is provided comprising selecting a patient having a vascular wound, and administering to the patient an effective amount of a bioactive agent capable of inducing endothelialization to the vascular lesion site.

In another embodiment of the method for induction of vascular wound healing of the present invention, the is a growth factor such as pleiotrophin, or an analog or derivative thereof.

In yet another embodiment of the present invention, the bioactive agent is delivered to the vascular lesion site by a drug-eluting stent, a drug-eluting angioplasty balloon or an injection catheter.

### BRIEF DESCRIPTION OF THE DRAWINGS

FIG. 1 depicts a schematic longitudinal cross-sectioned view through a substrate coated according to one embodiment of the invention.

FIG. 2 depicts a further exploded longitudinally cross-sectioned schematic view of finer detail of a porous bioactive coating useful according to the embodiment shown in the more general view of FIG. 1.

FIG. 3 depicts a longitudinally cross-sectioned view of another bioactive coated substrate surface according to a further embodiment of the invention.

FIG. 4 depicts a longitudinally cross-sectioned view of another bioactive coated substrate surface according to a further embodiment of the invention.

FIG. 5 depicts a schematic flow diagram of one method embodiment of the invention.

FIG. 6 depicts a schematic flow diagram of another method embodiment of the invention.

FIG. 7 depicts a cross-sectioned schematic view of an illustrative coating environment adapted for use according to various of the embodiments of the other FIGS.

FIG. 8 depicts a schematic partially longitudinally cross-sectioned view of a stented lumen such as a coronary or peripheral arterial vessel.

FIG. 9 depicts a schematic transversely cross-sectioned view through an illustrative strut of a stent adapted for use according to the mode shown in FIG. 8, and further with respect to various of the embodiments shown in the prior FIGS.

FIG. 10 depicts a flow chart diagram of one embodiment of the methods of the present invention.

FIG. 11 depicts an exemplary plasmid expressing the pleiotrophin gene according to the teachings of the present invention.

### DETAILED DESCRIPTION OF THE INVENTION

The present invention provides a medical device having a growth factor releasable disposed on the surface such that the growth factor promotes endothelialization of the device. The medical device of the present invention, such as an endolumenal stent, has a porous outer surface on the substrate that includes a plurality of pores wherein the pores contain a bioerodable or biodegradable material in combination with a bioactive agent, such as a growth factor. The stent is adapted to be positioned at a treatment site such that the pores are exposed to a body of a patient and the growth factor is released from the porous outer surface and promotes endothelialization of the treatment site.

There is a need to bias vascular wound healing after balloon angioplasty or stent placement toward re-endothelialization and away from neointimal hyperplasia. Towards that end, a molecular approach (Sousa et al., Circulation, 107:2274-2279, 2003), such as with the compositions and methods of the present invention, is proposed.

For the purposes of the present invention, bioactive agent, drug and therapeutic agent can be used interchangeably to refer to any organic, inorganic, or living agent that is biologically active or relevant. For example, a bioactive agent can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a bioactive agent. An example is a nanoparticle comprising a core with a drug and a coating on the core. Such nanoparticles can be post-loaded into pores or co-deposited with metal ions.

In one embodiment of the present invention, the bioactive agent is a growth factor. Non-limiting examples of growth factors suitable such as endothelial growth factor, or an analog or derivative thereof. In another embodiment of the present invention the bioactive agent is pleiotrophin, or an analog or derivative thereof.

As used herein “analogues” include compounds having structural similarity to another compound. For example, the anti-viral compound acyclovir is a nucleoside analogue and is structurally similar to the nucleoside guanosine which is derived from the base guanine. Thus acyclovir mimics guanosine (is “analogous with” biologically) and interferes with DNA synthesis by replacing (competing with) guanosine residues in the viral nucleic acid and prevents translation/transcription. Thus compounds having structural similarity to another (a parent compound) that mimic the biological or chemical activity of the parent compound are analogues. There are no minimum or maximum numbers of elemental or functional group substitutions required to qualify as an analogue as used herein providing the analogue is capable of mimicking, in some relevant fashion, either identically, complementary or competitively, with the biological or chemical properties of the parent compound. Analogues can be, and often are, derivatives of the parent compound (see “derivatives” infra). Analogues of the compounds disclosed herein may have equal, less or greater activity than their parent compounds.

As used herein a “derivative” is a compound made from (derived from), either naturally or synthetically, a parent compound. A derivative may be an analogue (see “analogue” supra) and thus may possess similar chemical or biological activity. However, as used herein, a derivative does not necessarily have to mimic the activity of the parent compound. There are no minimum or maximum numbers of elemental or functional group substitutions required to qualify as a derivative. As an example, the antiviral compound ganclovir is a derivative of acyclovir. Ganclovir has a different spectrum of anti-viral activity from that of acyclovir as well as different toxicological properties. Derivatives of the compounds disclosed herein may have equal, less, greater or no similar activity to their parent compounds.

Pleiotrophin is a secreted heparin-binding cytokine which stimulates mitogenesis and angiogenesis in vitro and stimulates endothelial cell migration and induces arteriogenesis in vivo (Christman K L et al., Molec Therapy 7(5):S231, 2003 U.S. Patent Application Publication No. 2003/0202960A1; and U.S. Patent Application Publication No. 2003/0185794A1, all of which are incorporated by reference herein for all they contain regarding pleiotrophin).

Pleiotrophin has previously been disclosed for wound healing of skin injuries such as ulceration, chronic wounds and surgical and other wounds. The present invention provides for pleiotrophin associated with vascular stents for vascular wound healing. Vascular wound healing refers to the treatment of vascular injury resulting from therapeutic vascular intervention (i.e. balloon angioplasty, atherectomy or stent placement), specifically, to achieve the proper balance of endothelial cell/smooth muscle cell growth after the injury.

A non-limiting example of vascular injury is restenosis. In one embodiment of the present invention, treatment of restenosis includes a pleiotrophin-eluding stent which promotes vascular wound healing by inducing endothelialization.

In another embodiment of the present invention, other vessels or lumens than blood vessels, such as, but not limited to, biliary duct, pancreatic duct, urethra, fallopian tubes, etc., are candidates for therapeutic wound healing of the vessel lining with pleiotrophin.

Pleiotrophin may be isolated from natural sources or by recombinant production by methods well-known in the art. Nucleic acid sequences and amino acid sequences of pleiotrophin are described in the art (Fang et al., 1992, J. Biol. Chem. 267:25889-25897; Li et al., 1990, Science 250:1690-1694; Lai et al., 1989, Biochem. Biophys. Res. Commun. 165: 1096-1103; Kadomatsu et al., 1988, Biochem. Biophys. Res. Commun. 151:1312-1318; Tornornura et al., 1990, J Biol. Chem. 265:10765; Vrios et al., 1991, Biochem. Biophys. Res. Commun. 175:617-624; and Li et al., 1992, J Biol. Chem., 267:26011-26016).

In one embodiment of the present invention, pleiotrophin is encoded by the amino acid sequence of SEQ ID NO. 1 and the nucleic acid sequence of SEQ ID NO 2.

| | | | | | | | | | | | | | | | | |
|---|---|---|---|---|---|---|---|---|---|---|---|---|---|---|---|---|
| Gly | Lys | Lys | Glu | Lys | Pro | Glu | Lys | Lys | Val | Lys | Lys | Ser | Asp | Cys | 15 | SEQ ID NO. 1 |
| GGG | AAG | AAA | GAG | AAA | CCA | GAA | AAA | AAA | GTG | AAG | AAG | TCT | GAC | TGT | 45 | SEQ ID NO. 2 |
| Gly | Glu | Trp | Gln | Trp | Ser | Val | Cys | Val | Pro | Thr | Ser | Gly | Asp | Cys | 30 | |
| GGA | GAA | TGG | CAG | TGG | AGT | GTG | TGT | GTG | CCC | ACC | AGT | GGA | GAC | TGT | 90 | |
| Gly | Leu | Gly | Thr | Arg | Glu | Gly | Thr | Arg | Thr | Gly | Ala | Glu | Cys | Lys | 45 | |
| GGG | CTG | GGC | ACA | CGG | GAG | GGC | ACT | CGG | ACT | GGA | GCT | GAG | TGC | AAG | 135 | |
| Gln | Thr | Met | Lys | Thr | Gln | Arg | Cys | Lys | Ile | Pro | Cys | Asn | Trp | Lys | 60 | |
| CAA | ACC | ATG | AAG | ACC | CAG | AGA | TGT | AAG | ATC | CCC | TGC | AAC | TGG | AAG | 180 | |
| Lys | Gln | Phe | Gly | Ala | Glu | Cys | Lys | Tyr | Gln | Phe | Gln | Ala | Trp | Gly | 75 | |
| AAG | CAA | TTT | GGC | GCG | GAG | TGC | AAA | TAC | CAG | TTC | CAG | GCC | TGG | GGA | 225 | |
| Gly | Cys | Asp | Leu | Asn | Thr | Ala | Leu | Lys | Thr | Arg | Thr | Gly | Ser | Leu | 90 | |
| GAA | TGT | GAC | CTG | AAC | ACA | GCC | CTG | AAG | ACC | AGA | ACT | GGA | AGT | CTG | 270 | |
| Lys | Arg | Ala | Leu | His | Asn | Ala | Glu | Cys | Gln | Lys | Thr | Val | Thr | Ile | 105 | |
| AAG | CGA | GCC | CTG | CAC | AAT | GCC | GAA | TGC | CAG | AAG | ACT | GTC | ACC | ATC | 315 | |
| Ser | Lys | Pro | Cys | Gly | Lys | Leu | Thr | Lys | Pro | Lys | Pro | Gln | Ala | Glu | 120 | |
| TCC | AAG | CCC | TGT | GGC | AAA | CTG | ACC | AAG | CCC | AAA | CCT | CAA | GCA | GAA | 360 | |
| Ser | Lys | Lys | Lys | Lys | Lys | Glu | GLy | Lys | Lys | Gln | Glu | Lys | Met | Leu | 135 | |
| TCT | AAG | AAG | AAG | AAA | AAG | GAA | GGC | AAG | AAA | CAG | GAG | AAG | ATG | CTG | 405 | |
| Asp | | | | | | | | | | | | | | | 136 | |
| GAT | | | | | | | | | | | | | | | 408 | |

The bovine cDNA and protein sequence and human cDNA and method of producing pleiotrophin protein, as well as expression vectors comprising the pleiotrophin DNA, have been previously disclosed in U.S. Pat. Nos. 6,448,381 and 6,511,823 and International Publication No. WO 99/53943 which are incorporated by reference herein for all they contain regarding the isolation and expression of pleiotrophin genes. Other pleiotrophin sequences include the sequences disclosed in Zhang et al., 1999, J. Biol. Chem. 274:12959. Other pleiotrophin sequences include recombinant polypeptides comprising one or more regions of a full-length pleiotrophin protein or gene.

In one embodiment of the present invention, the human pleiotrophin protein is incorporated in the matrix of the stent or in a stent coating where it is then released over a predetermined period of time for promoting vascular wound healing.

Pleiotrophin may be produced recombinantly and purified using any of a variety of methods available in the art (Sambrook et al., Molecular Cloning, A Laboratory Manual (2nd ed. 1989); Kriegler, Gene Transfer and Expression: A Laboratory Manual (1990); and Current Protocols in Molecular Biology Current Protocols in Molecular Biology (Ausubel et al., (eds.) 1997)).

Nucleic acid sequences encoding pleiotrophin can be typically cloned into intermediate vectors for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding pleiotrophin or production of protein. The nucleic acid encoding pleiotrophin can also be typically cloned into an expression vector, for administration to a plant cell, animal cell, a mammalian cell or a human cell, fungal cell, bacterial cell, or protozoa cell.

To obtain expression of a cloned gene or nucleic acid, pleiotrophin can be typically subcloned into an expression vector that contains a promoter to direct transcription. Suitable bacterial and eukaryotic promoters are well known in the art (Sambrook et al., Molecular Cloning, A Laboratory Manual (2nd ed. 1989); Kriegler, Gene Transfer and Expression: A Laboratory Manual (1990); and Current Protocols in Molecular Biology Current Protocols in Molecular Biology (Ausubel et al., (eds.) 1997). Eukaryotic and prokaryotic expression systems for bacteria, mammalian cells, yeast, and insect cells are well known in the art and are commercially available.

The promoter used to direct expression of a pleiotrophin nucleic acid depends on the particular application. For example, a strong constitutive promoter can be typically used for expression and purification of the pleiotrophin. In contrast, when pleiotrophin is administered in vivo for gene regulation, either a constitutive or an inducible promoter can be used, depending on the particular use of the pleiotrophin.

In one embodiment of the present invention, a stent is coated with a vector containing the nucleotide sequence encoding for a suitable growth factor. In another embodiment of the present invention, a stent is coated with a gene transfer vector containing the nucleotide sequence encoding for pleiotrophin (SEQ ID NO. 2).

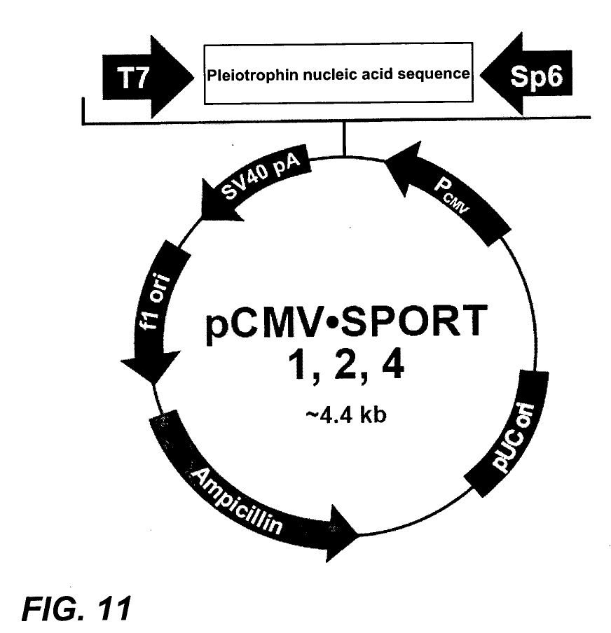

A number of vectors (viral or non-viral) known in the art may be used to achieve pleiotrophin protein expression in cardiovascular relevant sites. Non-limiting examples of gene transfer vectors include viral vectors and plasmids. An exemplary plasmid vector is depicted in FIG. 11. As the vector is released from the stent coating, it is able to enter into local endothelial cells which then secrete the pleiotrophin protein over a period of time and promote endothelialization of the stent, thus treating vascular injury. In one embodiment of the present invention, about 5 μg to about 1000 μg of the vector are coated on the stent, and in one particular embodiment 250 μg of vector are coated on the stent.

Any nucleic acid sequence encoding a pleiotrophin peptide and operably linked to suitable expression signals can be used within the context of the present invention. Whereas, the nucleic acid sequence can be operably linked to any suitable set of expression signals, in one embodiment, the expression of the DNA is under the control of the cytomegalovirus immediate early promoter.

Viral vectors, such as retroviruses, adenoviruses, adeno-associated viruses and herpes viruses, are often made up of two components, a modified viral genome and a coat structure surrounding it (Smith et al., 1995, Ann. Rev. Microbiol. 49:807-838). Alternatively, the vectors may be introduced in naked form or coated with proteins other than viral proteins. Most current vectors have coat structures similar to a wild-type virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line. Methods of making viral vectors comprising nucleic acid sequences encoding angiogenic factors are known in the art and may be applied to the present invention. (See, e.g., U.S. Patent application No. 20020019350, which is incorporated by reference herein for all it contains regarding methods of making viral vectors).

Nonviral nucleic acid vectors include, for example, plasmids, RNAs, antisense oligonucleotides (e.g., methylphosphonate or phosphorothiolate), polyamide nucleic acids, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Various efforts have been previously disclosed for achieving drug delivery from medical device surfaces by incorporating drugs into bioerodable or biodegradable materials, such as for example polymers, including but not limited to poly(lactide-co-glycolide), on the medical device surface. Certain such disclosures have included coating endolumenal stents with such composite materials. Additional specific disclosures have intended to construct such devices, including for example stents, from the combination drug-bioerodable matrix. Various material and design considerations are inherent in such applications, including in one regard providing the composite matrix with sufficient polymer component in the drug:polymer ratio to exhibit necessary integrity as a coating or mechanical scaffold. However, the biocompatibility of the polymer component is yet to be well settled and generally subject to scrutiny by leading molecular biologists and pathologists. This particularly applies to implants where the polymer components are released into vessel walls. Inflammation and foreign body reactions remain a concern principally due to the level of polymer burden within the erodable composite.

Drug delivery carrier vehicles such as coatings that incorporate drug into porous surfaces, e.g. porous polymers, also deliver drug via a relatively un-controlled diffusion gradient modality. Use of multiple layers of varying porosity or permeability to the drug elution have thus been disclosed in order to modify such elution characteristic to a desired time-based elution profile. There would be much benefit to provide the captured drug vehicle within such porous coatings in a manner that modifies the elution characteristic from a simple diffusion gradient.

Therefore, certain highly beneficial aspects of the invention are thus illustrated according to the detailed description and by reference to the accompanying figures as follows.

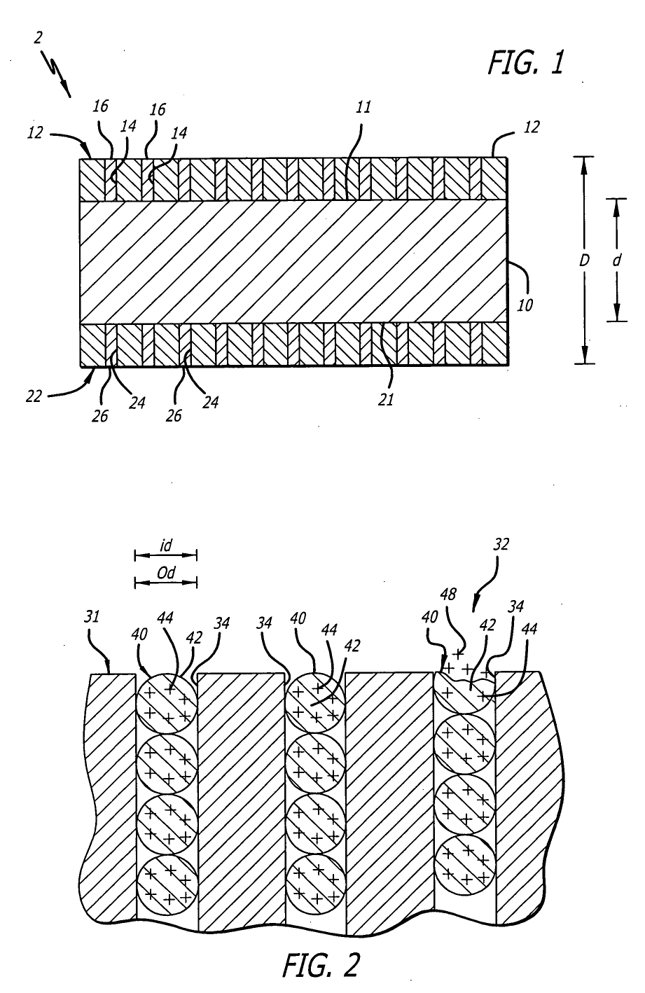

FIG. 1 shows a schematic view of a coated medical device implant 2 that illustrates certain aspects of the invention as follows. A substrate 10 is coated on a first surface 11 with a composite coating that includes a porous coating 12 with a plurality of pores 14 that are filled with a composite material 16. Composite material 16 includes a bioerodable material in combination with a bioactive agent. A second surface 21 is also coated similarly, with a composite coating having a porous coating material 22 with a plurality of pores 24 filled with bioerodable composite material 26 similar to that described for material 16.

Again, the various features shown and described are illustrative, and relative sizes and orientations, etc., of such components may be modified to suit a particular need, and in general are exaggerated for clarity, such as for example pores 14 which in particular beneficial embodiments are very small, e.g. micropores, and in further beneficial embodiments sub-micron sized nano-pores.

In various embodiments related to coronary stents for example, such sized pores will typically have a diameter of up to about 10 microns, most typically about 5 microns, and in some embodiments less than about 2 microns and even less than about 1 micron as nano-pores. Such porosity would be typically located on an underlying stent substrate having a diameter (d) of at least about 25 microns, generally up to about 55 microns, and whereas the resulting coated composite implant 2 would typically have illustrative cross-sectional diameters D between about 25 microns to about 75 microns, and generally between about 30 microns to about 50 microns. Coating thicknesses, (D minus d)/2, will typically range between about 2 microns to about 20 microns, and more typically between about 2 microns and about 10 microns. Again, all such dimensions are provided for further illustration of certain particular further embodiments, and other specific dimensions of the component parts, or differing relative comparative dimensions between them, are contemplated as would be apparent to one of ordinary skill.

FIG. 2 shows cross-sectional detail under a more exploded higher magnification schematic view of the porous composite coating similar to that adapted for use according to the FIG. 1 embodiment. More specifically, composite coating 32 includes a porous coating 31 that includes a plurality of pores 34 that each contains a plurality of composite particles 40. These composite particles 40 include a carrier material 42 in combination with a bioactive agent 44 according to certain broad aspects illustrated by the present embodiment. More specific to the particular embodiment shown, however, material 42 is a bioerodable material, which when exposed to the biological environment within a patient erodes to release the bioactive agent 48 into the surrounding environs. This is shown for illustration on the right side of FIG. 2.

Further to this particular embodiment, the particles 40 are shown with an outer diameter (od) that generally matches the inner diameter (id) of the pores 34. This is achieved for example by depositing these materials together, such as in an electroless electrochemical co-deposition process, wherein the formation of pores 34 may be self-defining around the particles 40 being co-deposited. Other methods may also be employed, such as, for example, by post-processing a pre-formed porous coating 31 with a later step of depositing particles 40, which may self-differentiate to particles equal to or less than the inner diameter id of the pores 34, keeping larger particles out. In this regard, even the co-deposition process of the electroless electrochemical embodiments may include differing relationships between pore sizes and the sizes of the particles deposited. Again, as noted above for FIG. 1, the relative sizes and conformations of the pores is provided in a particular relationship for clarity of illustration, and may vary depending upon the particular methods used or desired results.

The composite coatings just described by reference to FIGS. 1 and 2 were highly schematic and illustrative of a wide variety of particular coating schemes and resulting structures. Further specific embodiments follow for further illustration.

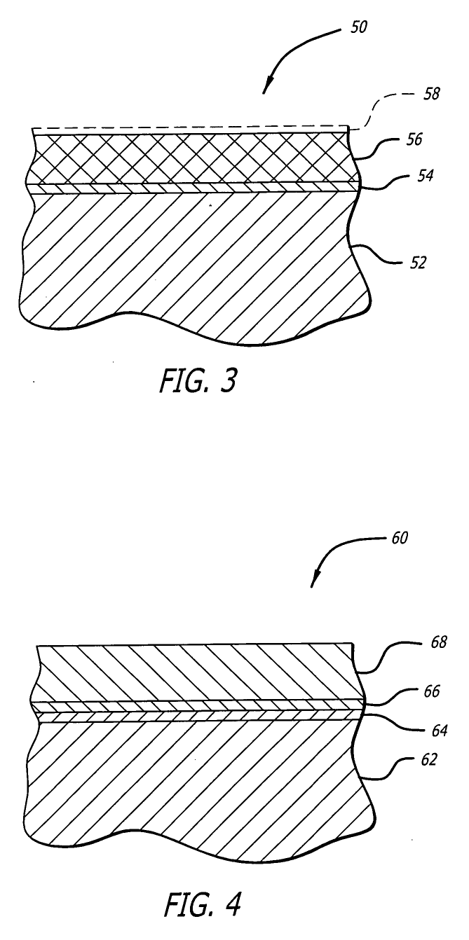

FIG. 3 shows a schematic cross-sectioned view of a further composite coating embodiment related to a coated medical device implant 50 as follows. Here, a substrate 52 is coated with a porous composite coating layer 56 which may be similar for example to that shown and described above by reference to FIGS. I and 2. In addition however, an additional coating layer 54 is shown located between the composite drug-loaded composite layer 56 and the underlying substrate 52. This additional layer 54 may be for example a tie layer in order to achieve optimal adhesion of the surface composite through the intended environment of use. In one particular further embodiment for example, the layer 54 may be an electroplated layer of nickel deposited for example onto a nickel-containing substrate 52 such as stainless steel or nickel-titanium, and overcoated by a composite drug coating layer of nickel-phosphorous providing micro- or nano-pores containing drug or drug-bioerodable composite material. This particular relationship of layers and corresponding materials is considered highly beneficial for example according to further detailed embodiments described in further detail below, and in particular beneficial relation to stents.

Accordingly, various different schemes of layered coating materials may be employed and remain consistent with the various broad objects and aspects of the present invention.

In a further example to illustrate, an outer layer 58 (shown in shadow in FIG. 3), may also be added over the porous composite coating layer 56 provided according the invention. This outer layer 58 may be, for example, another porous layer of material that modifies the elution of drugs from layer 56, or may be a second drug-carrying layer with a different material composition or dosing scheme from that in layer 56. In one particular example, layer 58 carries a different drug than layer 56. In another example, it carries a different drug dose than layer 56, either of the same drug, or of a different drug. Such outer layer 58 may also be bioerodable, either as a composite with a drug or as a protective covering that, once it erodes, initiates release of the drug from layer 56 with reduced encumbrance from the outer layer 58. This later scheme may, for example, protect the drug-coated device during delivery to the target lumen or body space.

A high-drug content bolus of elution may be related to the erosion of outer layer 58 as a bioerodable drug-carrying layer (either similar to layer 56 in general component parts or of other bioerodable drug-carrying vehicles available to one of ordinary skill), whereas a slower elution is related to erosion of the bioerodable contents of layer 56. Such schemes are consistent, for example, with the desired elution profiles of various anti-restenosis drugs where an initial, relatively high bolus. of drug is given within the first 24-48 hours after stent implantation, followed by slower elution over the ensuing period, e.g. between about 14 to about 28 days. For example, between about 20% to about 80%, and generally between about 20% and about 50%, of all drug may be delivered in the initial period, followed by the substantial remaining drug elution from the device over the subsequent period.

In another multi-layered composite coating embodiment shown in FIG. 4, a coated device implant 60 includes a substrate 62 coated with a composite coating layer 68 that elutes drugs, and two tie layers 64,66 between substrate 62 and outer layer 68. These two tie layers 64,66 may be, for example, a first layer 64 that is electroplated metal, followed by the second layer 66 that is an electrolessly electrochemically deposited layer of that metal in combination with a reducing agent of that metal. In specific embodiments for further exemplary illustration, substrate 62 is a nickel-containing metal such as stainless steel or nickel-titanium alloy, layer 64 is electroplated nickel, layer 66 is electrolessly electrochemically deposited nickel-phosphorous composite matrix, and layer 68 is electrolessly electrochemically deposited nickel-phosphorous composite matrix that is substantially porous and contains a bioactive agent, such as in a bioerodable composite matrix or particles as shown and described by reference to FIGS. 1 and 2 above. Moreover, an additional outer coat similar to layer 58 may also be added consistent with the objects and aspects described above for that layer by reference to FIG. 3.

It is to be further appreciated that various methods may be employed to produce the intended result according to certain aspects of the invention that include a bioerodable drug composite matrix within pores of a porous surface on a device implant.

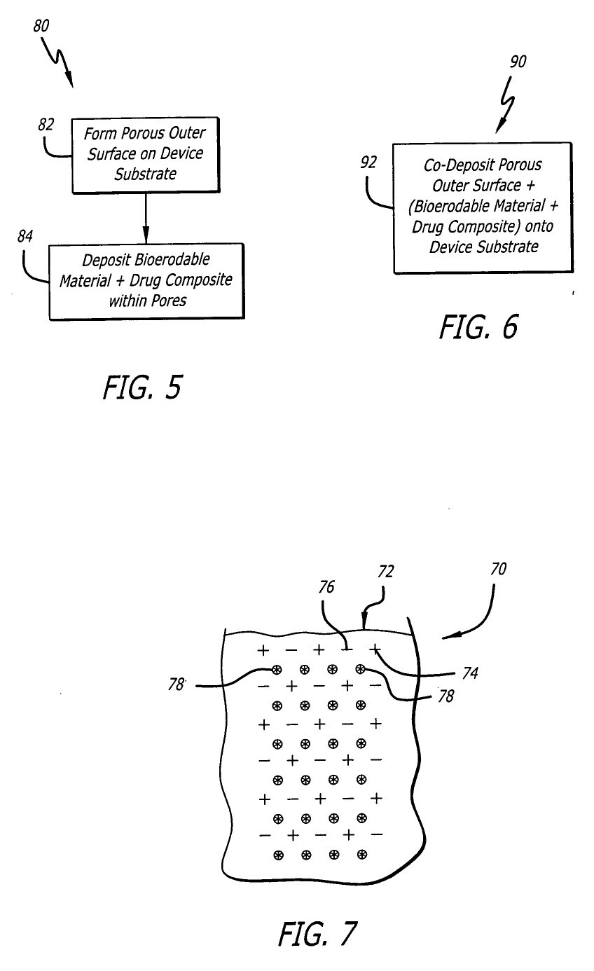

One particular example is shown in FIG. 5, wherein the coating method 80 includes a step 82 that forms a porous outer surface on a device substrate, followed by a step 84 that deposits a composite material with a bioerodable material in combination with a bioactive agent or drug within the pores of the porous outer surface first formed.

Such exemplary method may include various different porous surface treatments or coatings, e.g. polymers or sintered materials such as metal, ceramics, etc. Various different compositions and methods for depositing the bioerodable composite matrix into the pores are also contemplated, as would be apparent to one of ordinary skill based upon review of this disclosure and other available information. Such methods include, for example, exposing the porous coating to solvent solutions that cure, enlisting the aide of elevated pressures to deposit within the pores, fluid baths, atomized or nebulized environments of the depositing matrix material and/or its component parts, and including various particular preparations of micro- or nano-particles, etc.

In another highly beneficial mode shown schematically in FIG. 6, a method 90 may be used which simplifies such method to lesser steps, e.g. a co-deposition step 92 wherein a porous outer surface is co-deposited onto the desired device substrate together with a composite matrix of bioerodable material in combination with a drug.

Such latter method 90 described by reference to FIG. 6 is in particular a beneficial mode resulting from the use of electroless electrochemical co-deposition methods. Such methods and resulting materials and surface treatments are variously noted above for illustration purposes to the previous embodiments, and further described in more detail according to certain particular beneficial embodiments below.

As mentioned by reference to the embodiments of FIGS. 1-4 above, various modes of the invention are well suited for use with electrolessly electrochemically deposited porous composite coating matrix. This may be achieved according to a number of specific electroless electrochemical coating formulations and methods, with a variety of specific composite coating results.

A schematic view of a typical electroless electrochemical deposition bath is shown in FIG. 7 for illustration purposes. More specifically, an electroless electrochemical bath 72 is provided within a coating environment or container (e.g. suitable beaker, etc.), and includes among other component ingredients a soluble volume of metal ions 74 in a particular ratio combination with opposite valence ions or salts 76 that function as a reducing agent of the metal ions. Further included in the bath 72 is a suspended volume of micro- or nano-particles 78 that are composite materials of bioerodable material in combination with a drug. As elsewhere described herein, by exposing the properly active substrate surface to this bath formulation, the respective metal and reducing agent together form a composite matrix onto the exposed and active surface that captures the bioerodable drug composite particles within pores formed around those particles.

The result is a composite coating with the porous electrolessly electrochemically deposited metal-reducing agent composite and with the bioerodable-drug composite particles within those pores. A simple drying step after removal of the substrate (or otherwise removal of the catalytic ingredients such as under forced air or other inert gas) is often all that is required for a final result.

The foregoing description is a beneficial mode to provide a desired bioactive device implant surface, such as is consistent with the various embodiments described above with respect to FIGS. 1-4. However, further detail is provided below with respect to more particular beneficial coating modalities, and is to be appreciated in combination with these present embodiments among FIGS. 1-4, as well as FIGS. 5-7 (to the extent modified to provide drug loading separate from the porous coating formation with respect to FIG. 5). It is also to be appreciated that such further detailed embodiments with respect to specific coating methods and formulations provided below have further reaching benefits, other than in combination with the embodiments noted above.

Accordingly, a thin metal-based coating and a process for depositing the thin metal-based coating on implantable endolumenal medical devices is provided according to further aspects of the invention as follows.

In one particular mode, an improved method is provided for depositing a thin metal matrix onto the surface of an implantable device. The multiple step process deposits a composite thin metal matrix onto the device's surface. This multiple step process also includes one or more steps where a therapeutic or biologically active agent, or agents, is co-deposited with and within one or more thin metal films. The process is quite controllable, including with controlled variability of results, based on adjusting such parameters as temperature, pH, relative concentration of solution constituents, other additives or agents present in solution and time.

Specifically, the present invention makes use of the process of electroless electrochemical deposition to apply one or more layers of thin metal film, incorporating one or more drugs, onto the surface of an implantable device. According to one distinct benefit, electroless electrochemical deposition generally progresses as a self-assembling, autocatalytic process.

More specifically, in a further embodiment, the process of electroplating a surface is combined with electroless electrochemical deposition, in a multi-step approach. In one particular regard, such method has been observed to provide better adherence of the metallic matrix to the surface of the underlying device while also allowing for the incorporation of one or more bioactive agents with and within the coating matrix.

By one further more particular embodiment therefore, two solutions are prepared. The first solution is an electroplating or electrolytic solution or bath. The second solution is an electroless deposition solution or bath. The first bath is formed with a cathode (the device to be coated), and an electrolytic solution containing metal ions. The second bath is formed using metal salts, a solvent solution (e.g. aqueous environment), a reducing agent, and one or more bioactive agents to be incorporated into the coating matrix. Other materials are typically included in such second electroless electrochemical bath, as has been previously described and available to one of ordinary skill.

Prior to subjecting the device to the electrochemical processes described, the surface of the device is typically pre-treated in order to be appropriately prepared for suitable activity allowing for deposition thereon. Often, this aspect of the method includes de-oxidation of the surface, such as in the case of using substrate alloys such as stainless steel, cobalt-chromium, or nickel-titanium alloys that rapidly form generally non-reactive oxide layers on their surfaces exposed to oxygen rich environments. This may be accomplished for example by contacting or immersing the device in a pre-treatment bath, which may include for example organic or inorganic acids. For example, with regard to alloys such as stainless steel, nickel-titanium, or cobalt-chromium an acid bath (or series thereof) may be used that includes one or a combination of inorganic acids such as hydrochloric acid (HCl), nitric acid (HNO₃), or hydrofluoric acid (HF). Other methods of cleaning the surface can include molten salts, mechanical removal, alkaline cleaning, or any other suitable method that provides a clean, coatable surface. This initial step generally serves to clean the surface and etch the surface thereby removing any resident oxide layers on the structure and pitting the surface to improve subsequent adherence of the coating to the device.

The device is then rinsed with, for example, deionized water or deionized and distilled water, although, other suitable liquids or gasses could be used to remove any possible impurities from the surface. After rinsing, the implantable structure to be coated is immersed in the first bath. A current is then applied across the device causing the metal ions to move to the device and plate the surface. This electroplating step causes an intermediate or “strike” layer to be formed on the surface of the device. Metal ions for this first bath are typically chosen to be compatible with the material making up the device itself. For example, if the underlying structure is made of cobalt chrome, cobalt ions are preferred. It has been observed that this strike layer improves overall adherence of the coating to the implantable device as well as increasing the rate of deposition or efficiency of the second, electroless film. The device is subsequently removed from the first bath, and may be rinsed again with water prior to immersion into the second bath.

The device is then immersed in the second, electroless bath at a controlled temperature and pH value. In this step, metal ions, the reducing agent, and the one or more drugs are simultaneously and substantially uniformly, co-deposited on the struck surface of the device. After immersion in this second bath, a bioactive composite metallic matrix has been formed on the surface of the device. The device is removed from the second bath and allowed to dry.

By this deposition process, any suitable structure can be coated. The device can be porous or solid, flexible or rigid, have a planar or non-planar surface. Accordingly, in some embodiments the device could be a stent, a pellet, a pill, a seed, an electrode, a coil, etc. The device to be coated may be formed of any suitable material such as, metal, metal alloy, ceramic, polymer, glass, etc.

Any suitable source of metal ions can be used for the first electrolytic bath. Typically, such metal ions are derived from metal salts which dissociate from one another in solution. Such salts, and therefore ions, are well known in the field of electrolytic deposition and can be chosen by those of ordinary skill in this art. Examples of suitable metal ions depend on the underlying device to be coated, but does include ions of nickel, copper, gold, cobalt, silver, palladium, platinum, etc., and alloys thereof. Different types of salts can be used if it is desired to strike a metal alloy matrix on the surface of the device.

Similarly, any suitable source of metal ions can be used for the second electroless electrochemical deposition bath. Such are also typically derived from metal salts. Examples of such suitable sources depend on the underlying device to be coated and are well known in the field of electroless electrochemical deposition and can be selected by those of ordinary skill in this art.

The electroless electrochemical solution also generally includes a reducing agent and may include complexing agents, buffers and stabilizers. The reducing agent reduces the oxidation state of the metal ions in solution such that the metal ions deposit on the surface of the device as metal. Complexing agents are used to hold the metal in solution. Buffers and stabilizers are used to increase bath life and improve stability of the bath. Buffers are also used to control the pH of the solution. Stabilizers are also used to keep the solution homogeneous. Examples of such complexing agents, buffers and stabilizers are well known in the field of electroless electrochemical deposition and can be selected by those of ordinary skill in this art.

Concerning the bioactive agent to be co-deposited, any such agent, agents, or combinations thereof can be deposited within the coating depending on the condition to be treated, response desired, or tissue into which the device is to be introduced. Agents which can be coated onto the surface of the device in accordance with the invention include for example the following compounds; organic, inorganic, water soluble, water insoluble, hydrophobic, hydrophilic, lipophilic, large molecules, small molecules, proteins, anti-proliferatives, anti-inflammatory, anti-thrombogenetic, anti-biotic, anti-viral, hormones, growth factors, immunosuppressants, chemotherapeutics, etc. A preferred bioactive agent is pleiotrophin.

These bioactive agents are co-deposited or captured within the electroless electrochemically deposited layer, diffuse out or are released from the coating via pores formed in the coating by the coating process itself. The metal composite matrix forms pores between self-assembling grains as they meet and grow on the surface being coated. This porosity, or the extent and nature of these pores, is a property that is readily manipulated according to proven methods well known to those of ordinary skill in this art.

With regard to the first electroplating bath, in another embodiment of the invention, one or more intermediate layers can be struck on the surface of the device. This can improve the efficiency of the subsequent electroless electrochemical coating step.

Likewise, with regard to the second electroless electrochemical bath, one or more films can be coated onto the surface of the device. Furthermore, multiple electroless electrochemical baths can be used such that not all these baths co-deposit one or more bioactive agents. For example, after the electroplating step, a first electroless electrochemical bath without any bioactive agents can be employed to place a first electroless coating onto the surface of the device. The device can then be transferred to a second electroless bath containing one or more bioactive agents in solution. This can improve the efficiency of the step involving co-deposition of the metal ions, reducing agent and one or more bioactive agents.

Moreover, multiple electroless baths can be prepared containing and co-depositing different bioactive agents in each coating layer. In addition, an electroless bath, not containing any bioactive agents, can be applied as a top coat to modify or control the release of bioactive agents from an inner layer or layers.

The scope of the present invention, is not at all limited by this description, though these descriptions are independently considered highly beneficial and useful. Nor is the implantable device limited to a stent, though again such application and resulting composite device is independently beneficial and useful. In particular, the intricate microstructures of stents and ability to uniformly, controllably, and robustly coat such microstructures with high degrees of integrity through stent expansion is considered of particular competitive benefit compared with other alternative coating modalities. Nonetheless, with respect to certain broad aspects contemplated hereunder, these descriptions are illustrative of a manner in which such aspects of the invention can be practiced.

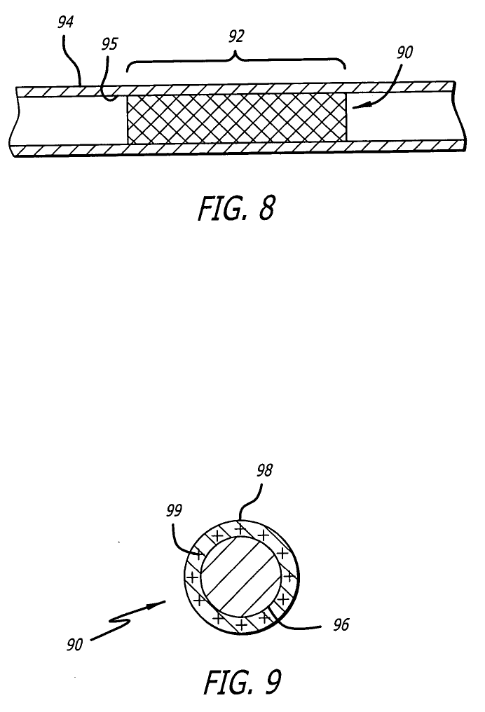

Moreover, with respect to stent applications of the various embodiments herein described, illustrative examples are variously shown in FIGS. 8 and 9. More specifically, FIG. 8 shows a stent 90 implanted along a stenting segment 92 of a lumen 94, such as a coronary or peripheral artery vessel, in an expanded configuration that engages the vessel wall 95 and as a support scaffold holds it open.

A cross-section of an illustrative stent strut is shown in FIG. 9, and includes an underlying scaffold 96 surrounded by an outer coating 98 that includes a bioactive agent 99. As previously described, the scaffold 96 may be of many different specific types of material, but in general typically are metal alloys such as for example stainless steel, nickel-titanium, or cobalt-chrome. With respect to the various coating examples and embodiments herein described, typically beneficial choices for electroless electrochemical coating deposition include nickel-phosphorous composites for the nickel-rich stainless steel and nickel-titanium alloys, whereas a cobalt-phosphorous may be beneficially chosen for the cobalt-chrome alloys. Moreover, the electroplated strike layers for such coatings, if utilized, will often be electroplated nickel for such nickel-rich alloys and nickel-based electroless outer coating composite, or possibly electroplated cobalt or chrome for such cobalt or chrome-containing cobalt-chrome alloys (or with respect to other such applications of cobalt-phosphorous electroless depositions).

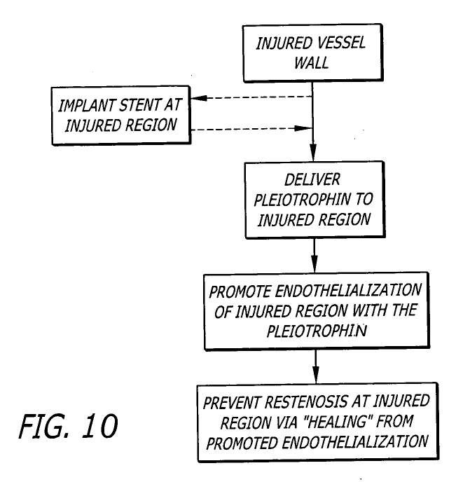

FIG. 10 depicts a flow diagram of one embodiment of the present invention for delivering pleiotrophin, or an analog or derivative thereof, to an injured region of a blood vessel in order to promote endothelialization and thus prevent restenosis. In another embodiment of the present invention, the delivery of pleiotrophin is in conjunction with stenting, shown in dashed line, wherein the stenting may be the procedure by which the injury has been made or is adjunctive thereto, e.g., after atherectomy or predilation via angioplasty.

In yet. another embodiment of the present invention, the growth factor delivery may be accomplished via local delivery of the growth factor directly to the vessel treatment site, such as through an endhole or sidehole injection catheter, or via a needle injection catheter in the presence or the absence of a stent at the treatment site. An exemplary non-limiting needle injection catheter suitable for use with the present invention is the MicroSyringe™, a product of EndoBionics, Inc. (San Leandro, Calif.).

Pleiotrophin is herein described as a highly beneficial aspect of the invention, though other analogs or derivatives thereof may be used and contemplated within the intended scope of various aspects of the invention. For example, similar bioactivity may be achieved with modifications to the pleiotrophin molecule without departing from the intended scope of the invention. In one embodiment, pleiotrophin active sites and amino acid sequences associated therewith may be incorporated onto other molecules chains to provide further embodiments of the present invention.

### EXAMPLE 1

### Delivery of Pleiotrophin Gene Induces Neovasculature Formation in Ischemic Myocardium

The pleiotrophin (PTN) gene nucleic acid sequence (SEQ ID NO. 2) was inserted into the cloning site of a PCMV plasmid (FIG. 11) and mammalian cells transfected with the plasmid are capable of producing and secreting pleiotrophin. The pleiotrophin produced from these cells induced the proliferation of serum-starved SW13 cells (non-epithelial, adrenal cortex-derived human cells), indicating that it was biologically active.

The pCMV-PTN plasmid (250 μg) was injected directly into the infarcted myocardium of female Sprague-Dawley rats (n=3) following occlusion of their left anterior descending portion of the left coronary artery for 17 minutes. Another set of rats (n=2) was injected with a control plasmid (pCMV-βgal). The rats were sacrificed after a minimum of five weeks. The hearts were rapidly excised, fresh frozen and sectioned into 10 μm slices. Five sections from each heart were stained for capillaries using a Griffonia simplicifolia lectin which binds to a carbohydrate domain on endothelial cells and another five sections were stained for arterioles using an anti-smooth muscle actin antibody. Injection of the PCMV-PTN plasmid induced formation of neovasculature compared to the control plasmid. The capillary density following delivery of the pCMV-PTN plasmid significantly increased to 1258 ±157 capillaries/mm² compared to 782±166 capillaries/mm² with control plasmid. The arteriole density also increased to 11±2 arterioles/mm² compared to 4±0 arterioles/mm² for the control plasmid. Thus, delivery of the pleiotrophin gene to ischemic myocardium results in production of pleiotrophin protein and promotes angiogenesis.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An endolumenal stent system for promoting endothelialization of vascular injury sites, comprising:
an endolumenal stent;
a porous surface on the endolumenal stent having a plurality of pores; and
a composite material located within each of the pores and comprising a bioerodable material in combination with a therapeutically effective amount of a bioactive agent.

2. The endolumenal stent system of claim 1, wherein the composite material comprises a plurality of particles.

3. The endolumenal stent system of claim 2, wherein the particles comprise a bioerodable polymer in combination with said bioactive agent.

4. The endolumenal stent system of claim 1, wherein said bioactive agent is a growth factor.

5. The endolumenal stent system of claim 4, wherein said growth factor is a biologically active endothelial growth factor.

6. The endolumenal stent system of claim 4, wherein said growth factor is pleiotrophin, or an analog or derivative thereof.

7. The endolumenal stent system of claim 6 wherein said pleiotrophin has the amino acid sequence of SEQ ID NO. 2 or a portion thereof.

8. The endolumenal stent system of claim 6 wherein said pleiotrophin has the nucleotide sequence of SEQ ID NO. 1 or a portion thereof.

9. The endolumenal stent system of claim 6 wherein said pleiotrophin is formulated as a protein or peptide.

10. The endolumenal stent system of claim 6 wherein said pleiotrophin is formulated as a gene transfer vector encoding a protein or a peptide.

11. The endolumenal stent system of claim 10 wherein said gene transfer vector is a plasmid.

12. A method for promoting vascular wound healing by induction of endothelialization of a vascular lesion comprising:
selecting a patient having a vascular wound; and
administering to said patient an effective amount of a bioactive agent capable of inducing endothelialization to the vascular lesion site.

13. The method of claim 12 wherein said bioactive agent is a growth factor.

14. The method of claim 13 wherein said growth factor is a biologically active endothelial growth factor.

15. The method of claim 13 wherein said growth factor is pleiotrophin, or an analog or derivative thereof.

16. The method of claim 15 wherein said pleiotrophin is formulated as a protein or peptide.

17. The method of claim 15 wherein said pleiotrophin is formulated as a gene transfer vector encoding a protein or a peptide.

18. The method of claim 17 wherein said gene transfer vector is a plasmid.

19. The method of claim 12 wherein said bioactive agent is delivered to said vascular lesion site by a drug-eluting stent.

20. The method of claim 12 wherein said bioactive agent is delivered to said vascular lesion site by a drug-eluting angioplasty balloon.

21. The method of claim 12 wherein said bioactive agent is delivered to said vascular lesion site by an injection catheter.


SEQUENCE LISTING (2 sequences)

SEQ ID NO: 1 - protein, 136 aa, Homo sapiens
GKKEKPEKKVKKSDCGEWQWSVCVPTSGDCGLGTREGTRTGAECKQTMKTQRCKIPCNWKKQFGAECKYQFQAWGGCDLNTALKTRTGSLKRALHNAECQKTVTISKPCGKLTKPKPQAESKKKKKEGKKQEKMLD

SEQ ID NO: 2 - DNA, 408 bp, Homo sapiens
gggaagaaagagaaaccagaaaaaaaagtgaagaagtctgactgtggagaatggcagtggagtgtgtgtgtgcccaccagtggagactgtgggctgggcacacgggagggcactcggactggagctgagtgcaagcaaaccatgaagacccagagatgtaagatcccctgcaactggaagaagcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggat